SEQUENCE LISTING (12 sequences)

SEQ ID NO: 1 - DNA, 1788 bp, Triticum aestivum
  CDS (1)..(1788)
tcccccgccgccacctccgccgcgcctcccgcaaccgcgctccggccctggggcccgtccgagccccgcaagggcgccgacatcctcgtcgaggcgctcgagcgctgcggcatcgtcgacgtcttcgcctaccccggcggcgcctccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgccgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttccttgcatcctctggccgcccggggccggtgctagttgatatccccaaggacatccagcagcagatggctgtgcccgtctgggacactccaatgagtttgccagggtacatcgcccgcctgcccaagccaccatctactgaatcgcttgagcaggtcctgcgtctggttggcgagtcacggcgcccaattctgtatgttggtggtggctgcgctgcgtctggcgaggagttgcgccgctttgttgagcttactgggattccagttacaactactctgatgggccttggcaacttccccagcgacgacccactgtctctgcgcatgcttgggatgcatggcactgtgtatgcaaattatgcagtagataaggctgacctgttgctcgcatttggtgtgcggtttgatgatcgtgtgactgggaaaatcgaggcttttgcaagcaggtccaagattgtgcacattgacattgacccagctgagattggcaagaacaagcagccacatgtctccatttgtgcagatgttaagcttgctttacaggggttgaatgatctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctgggaatggtggtgcagtgggaggataggttttacaaggccaatcgggcgcacacataccttggcaacccagaaaatgagagtgagatatatccagattttgtgacgattgctaaaggattcaacgttccagcagttcgagtgacgaagaagagcgaagtcactgcagcaatcaagaagatgcttgagaccccagggccatacttgttggatatcatagtcccgcatcaggagcacgtgctgcctatgatcccaagcggtggtgctttcaaggacatgatcatggagggtgatggcaggacctcgtac

SEQ ID NO: 2 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 3 - DNA, 1788 bp, Triticum aestivum
  CDS (1)..(1788)
tcccccgccgccacctccgccgcgcctcccgccaccgcgctccggccgtggggcccctccgagccccgcaagggcgccgacatcctcgtcgaggcgctggagcgctgcggcatcgtcgacgtcttcgcctaccctggcggcgcgtccatggagatccaccaggcgctgacgcgctcgccagtcatcaccaaccacctcttccgccacgagcagggggaggcgttcgcggcgtccgggtacgcccgcgcgtccggccgcgtcggcgtctgcgtcgccacctccggcccgggggccaccaacctcgtctccgcgctcgccgacgctctcctcgactccatccccatggtcgccatcacgggccaggtcccccgccgcatgatcggcacggatgcgttccaggagacgcccatcgtggaggtcacgcgctccatcaccaagcacaactacctggtccttgacgtggaggatatcccccgcgtcatccaggaagccttcttcctcgcatcctctggccgcccggggccggtgctggttgatatccccaaggacatccagcagcagatggctgtgcctgtctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatctggtgaggagttgcgccgctttgttgagctcactgggattccagttacaactactcttatgggccttggcaacttccccagtgacgacccactgtctctgcgcatgctggggatgcatggcactgtgtatgcaaattatgcagtagataaggctgacctgttgcttgcatttggtgtgcggtttgatgatcgtgtgaccgggaaaatcgaggcttttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggtgaggccatcccgccgcaatatgctatccaggtactggatgagctgacaaaaggggaggcgatcattgccaccggtgttgggcagcatcagatgtgggcggctcagtattacacttacaagcggccacggcagtggctgtcttcatccggtttgggtgcaatgggatttgggttgccagctgcagctggcgctgctgtggccaacccaggtgttacagttgttgacattgatggggatggtagtttcctcatgaacattcaggagttggcgttgatccgtattgagaacctcccagtgaaggtgatgatattgaacaaccagcatctgggaatggtggtgcagtgggaggataggttttacaaggccaaccgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcattgtcccgcatcaggagcacgtgctgcctatgatcccaagcggtggtgcttttaaggacatgatcatggagggtgatggcaggacctcgtac

SEQ ID NO: 4 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 5 - DNA, 1788 bp, Triticum aestivum
  CDS (1)..(1788)
tcccccgccgccacctccgccgcgccccccgccaccgcgctccggccctggggcccgtccgagccccgcaagggcgccgacatcctcgtcgaggcgctcgagcgctgcggcatcgtcgacgtattcgcctaccccggcggcgcgtccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgctgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttcctcgcgtcctctggccgcccggggccggtgctggttgatatccccaaggatatccagcagcagatggccgtgcctatctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatccggcgaggagttgcgccgctttgttgagctcactgggattccggttacaactactctgatgggccttggcaacttccccagcgacgacccactgtctctgcgcatgcttgggatgcatggcactgtgtatgcaaattatgcagtcgataaggctgacctgttgcttgcatttggtgtgcggtttgatgatcgcgtgactgggaaaatcgaggcctttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgctactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggagatggtagtttcctcatgaacattcaggagttggcattgatccgtattgagaacctccctgtgaaggtgatgatattgaacaaccagcatctgggaatggtggtgcaatgggaggataggttttacaaggccaatcgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcatcgtcccgcatcaggagcacgtgctgcctatgatcccaagcggtggtgctttcaaggacatgatcatggagggtgatggcaggacctcgtac

SEQ ID NO: 6 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPIWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 7 - DNA, 1788 bp, Triticum aestivum
  CDS (1)..(1788)
tcccccgccgccacctccgccgcgcctcccgcaaccgcgctccggccctggggcccgtccgagccccgcaagggcgccgacatcctcgtcgaggcgctcgagcgctgcggcatcgtcgacgtcttcgcctaccccggcggcgcctccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgccgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttccttgcatcctctggccgcccggggccggtgctagttgatatccccaaggacatccagcagcagatggctgtgcccgtctgggacactccaatgagtttgccagggtacatcgcccgcctgcccaagccaccatctactgaatcgcttgagcaggtcctgcgtctggttggcgagtcacggcgcccaattctgtatgttggtggtggctgcgctgcgtctggcgaggagttgcgccgctttgttgagcttactgggattccagttacaactactctgatgggccttggcaacttccccagcgacgacccactgtctctgcgcatgcttgggatgcatggcactgtgtatgcaaattatgcagtagataaggctgacctgttgctcgcatttggtgtgcggtttgatgatcgtgtgactgggaaaatcgaggcttttgcaagcaggtccaagattgtgcacattgacattgacccagctgagattggcaagaacaagcagccacatgtctccatttgtgcagatgttaagcttgctttacaggggttgaatgatctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctgggaatggtggtgcagtgggaggataggttttacaaggccaatcgggcgcacacataccttggcaacccagaaaatgagagtgagatatatccagattttgtgacgattgctaaaggattcaacgttccagcagttcgagtgacgaagaagagcgaagtcactgcagcaatcaagaagatgcttgagaccccagggccatacttgttggatatcatagtcccgcatcaggagcacgtgctgcctatgatcccaaacggtggtgctttcaaggacatgatcatggagggtgatggcaggacctcgtac

SEQ ID NO: 8 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPNGGAFKDMIMEGDGRTSY

SEQ ID NO: 9 - DNA, 1788 bp, Triticum aestivum
  CDS (1)..(1788)
tcccccgccgccacctccgccgcgcctcccgccaccgcgctccggccgtggggcccctccgagccccgcaagggcgccgacatcctcgtcgaggcgctggagcgctgcggcatcgtcgacgtcttcgcctaccctggcggcgcgtccatggagatccaccaggcgctgacgcgctcgccagtcatcaccaaccacctcttccgccacgagcagggggaggcgttcgcggcgtccgggtacgcccgcgcgtccggccgcgtcggcgtctgcgtcgccacctccggcccgggggccaccaacctcgtctccgcgctcgccgacgctctcctcgactccatccccatggtcgccatcacgggccaggtcccccgccgcatgatcggcacggatgcgttccaggagacgcccatcgtggaggtcacgcgctccatcaccaagcacaactacctggtccttgacgtggaggatatcccccgcgtcatccaggaagccttcttcctcgcatcctctggccgcccggggccggtgctggttgatatccccaaggacatccagcagcagatggctgtgcctgtctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatctggtgaggagttgcgccgctttgttgagctcactgggattccagttacaactactcttatgggccttggcaacttccccagtgacgacccactgtctctgcgcatgctggggatgcatggcactgtgtatgcaaattatgcagtagataaggctgacctgttgcttgcatttggtgtgcggtttgatgatcgtgtgaccgggaaaatcgaggcttttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggtgaggccatcccgccgcaatatgctatccaggtactggatgagctgacaaaaggggaggcgatcattgccaccggtgttgggcagcatcagatgtgggcggctcagtattacacttacaagcggccacggcagtggctgtcttcatccggtttgggtgcaatgggatttgggttgccagctgcagctggcgctgctgtggccaacccaggtgttacagttgttgacattgatggggatggtagtttcctcatgaacattcaggagttggcgttgatccgtattgagaacctcccagtgaaggtgatgatattgaacaaccagcatctgggaatggtggtgcagtgggaggataggttttacaaggccaaccgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcattgtcccgcatcaggagcacgtgctgcctatgatcccaaacggtggtgcttttaaggacatgatcatggagggtgatggcaggacctcgtac

SEQ ID NO: 10 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPNGGAFKDMIMEGDGRTSY

SEQ ID NO: 11 - DNA, 1788 bp, Triticum aestivum
  CDS (1)..(1788)
tcccccgccgccacctccgccgcgccccccgccaccgcgctccggccctggggcccgtccgagccccgcaagggcgccgacatcctcgtcgaggcgctcgagcgctgcggcatcgtcgacgtattcgcctaccccggcggcgcgtccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgctgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttcctcgcgtcctctggccgcccggggccggtgctggttgatatccccaaggatatccagcagcagatggccgtgcctatctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatccggcgaggagttgcgccgctttgttgagctcactgggattccggttacaactactctgatgggccttggcaacttccccagcgacgacccactgtctctgcgcatgcttgggatgcatggcactgtgtatgcaaattatgcagtcgataaggctgacctgttgcttgcatttggtgtgcggtttgatgatcgcgtgactgggaaaatcgaggcctttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgctactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggagatggtagtttcctcatgaacattcaggagttggcattgatccgtattgagaacctccctgtgaaggtgatgatattgaacaaccagcatctgggaatggtggtgcaatgggaggataggttttacaaggccaatcgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcatcgtcccgcatcaggagcacgtgctgcctatgatcccaaacggtggtgctttcaaggacatgatcatggagggtgatggcaggacctcgtac

SEQ ID NO: 12 - protein, 596 aa, Triticum aestivum
SPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPIWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPNGGAFKDMIMEGDGRTSY

(19) United States
(12) Patent Application Publication
(10) Pub. No.: US 2006/0010514 A1
(43) Pub. Date: Jan. 12, 2006

(54) Polynucleotides encoding mature AHASL proteins for creating imidazolinone-tolerant plants

(75) Inventors: Iwona Birk, Raleigh, NC (US); Bijay K. Singh, Cary, NC (US); Gregory B. Parker, Ames, IA (US)

Correspondence Address: ALSTON & BIRD LLP;BASF CORPORATION, BANK OF AMERICA PLAZA, 101 SOUTH TRYON STREET, SUITE 4000, CHARLOTTE, NC, 28280-4000, US

(73) Assignee: BASF Plant Science GmbH, Ludwigshafen (DE)

(21) Appl. No.: 11/152,903

(22) Filed: Jun. 15, 2005

Related U.S. Application Data
(60) Provisional application No. 60/580,021, filed on Jun. 16, 2004.

(51) Int. Cl. A01H 1/00; C12N 15/82

(52) U.S. Cl. 800/278

(57) ABSTRACT

Isolated polynucleotide molecules that encode mature, wild-type and imidazolinone-tolerant acetohydroxyacid synthase large subunit (AHASL) polypeptides, and the amino acid sequences encoding these polypeptides, are described. Expression cassettes and transformation vectors comprising the polynucleotide molecules of the invention, as well as plants and host cells transformed with the polynucleotide molecules, expression cassettes, and transformation vectors, are described. Methods of using the polynucleotide molecules to enhance the resistance of plants to herbicides, and methods for controlling weeds in the vicinity of such plants are also described.

### CROSS-REFERENCE TO RELATED APPLICATIONS

This application claims the benefit of U.S. Provisional Application No. 60/580,021, filed Jun. 16, 2004, which is incorporated herein in its entirety.

### FIELD OF THE INVENTION

This invention relates to the field of plant molecular biology, particularly to novel nucleotide sequences that encode wheat acetohydroxyacid synthase large subunit enzymes (hereinafter AHASL) and that can be used to create herbicide-tolerant plants.

### BACKGROUND OF THE INVENTION

Acetohydroxyacid synthase (AHAS; EC 4.1.3.18, also known as acetolactate synthase or ALS), is the first enzyme that catalyzes the biochemical synthesis of the branched chain amino acids valine, leucine and isoleucine (Singh (1999) “Biosynthesis of valine, leucine and isoleucine,” in Plant Amino Acid, Singh, B. K., ed., Marcel Dekker Inc. New York, N.Y., pp. 227-247). AHAS is the site of action of four structurally diverse herbicide families including the sulfonylureas (LaRossa and Falco (1984) Trends Biotechnol. 2:158-161), the imidazolinones (Shaner et al. (1984) Plant Physiol. 76:545-546), the triazolopyrimidines (Subramanian and Gerwick (1989) “Inhibition of acetolactate synthase by triazolopyrimidines,” in Biocatalysis in Agricultural Biotechnology, Whitaker, J. R. and Sonnet, P. E. eds., ACS Symposium Series, American Chemical Society, Washington, D.C., pp. 277-288), and the pyrimidyloxybenzoates (Subramanian et al. (1990) Plant Physiol. 94: 239-244.). Imidazolinone and sulfonylurea herbicides are widely used in modern agriculture due to their effectiveness at very low application rates and relative non-toxicity in animals. By inhibiting AHAS activity, these families of herbicides prevent further growth and development of susceptible plants including many weed species. Several examples of commercially available imidazolinone herbicides are PURSUIT® (imazethapyr), SCEPTER® (imazaquin) and ARSENAL® (imazapyr). Examples of sulfonylurea herbicides are chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfiuon, fluzasulfuron, imazosulfuron, pyrazosulfuron ethyl and halosulfuron.

Due to their high effectiveness and low-toxicity, imidazolinone herbicides are favored for application by spraying over the top of a wide area of vegetation. The ability to spray an herbicide over the top of a wide range of vegetation decreases the costs associated with plantation establishment and maintenance, and decreases the need for site preparation prior to use of such chemicals. Spraying over the top of a desired tolerant species also results in the ability to achieve maximum yield potential of the desired species due to the absence of competitive species. However, the ability to use such spray-over techniques is dependent upon the presence of imidazolinone-resistant species of the desired vegetation in the spray over area.

Among the major agricultural crops, some leguminous species such as soybean are naturally resistant to imidazolinone herbicides due to their ability to rapidly metabolize the herbicide compounds (Shaner and Robinson (1985) Weed Sci. 33:469-471). Other crops such as corn (Newhouse et al. (1992) Plant Physiol. 100:882886) and rice (Barrette et al. (1989) Crop Safeners for Herbicides, Academic Press, New York, pp. 195-220) are somewhat susceptible to imidazolinone herbicides. The differential sensitivity to the imidazolinone herbicides is dependent on the chemical nature of the particular herbicide and differential metabolism of the compound from a toxic to a non-toxic form in each plant (Shaner et al. (1984) Plant Physiol. 76:545-546; Brown et al., (1987) Pestic. Biochem. Physiol. 27:24-29). Other plant physiological differences such as absorption and translocation also play an important role in sensitivity (Shaner and Robinson (1985) Weed Sci. 33:469-471).

Crop cultivars resistant to imidazolinones, sulfonylureas and triazolopyrimidines have been successfully produced using seed, microspore, pollen, and callus mutagenesis in Zea mays, Arabidopsis thaliana, Brassica napus, Glycine max, and Nicotiana tabacum (Sebastian et al. (1989) Crop Sci. 29:1403-1408; Swanson et al., 1989 Theor. Appl. Genet. 78:525-530; Newhouse et al. (1991) Theor. Appl. Genet. 83:65-70; Sathasivan et al. (1991) Plant Physiol. 97:1044-1050; Mourand et al. (1993) J. Heredity 84:91-96). In all cases, a single, partially dominant nuclear gene conferred resistance. Four imidazolinone resistant wheat plants were also previously isolated following seed mutagenesis of Triticum aestivum L. cv. Fidel (Newhouse et al. (1992) Plant Physiol. 100:882-886). Inheritance studies confirmed that a single, partially dominant gene conferred resistance. Based on allelic studies, the authors concluded that the mutations in the four identified lines were located at the same locus. One of the Fidel cultivar resistance genes was designated FS-4 (Newhouse et al. (1992) Plant Physiol. 100:882-886).

Computer-based modeling of the three dimensional conformation of the AHAS-inhibitor complex predicts several amino acids in the proposed inhibitor binding pocket as sites where induced mutations would likely confer selective resistance to imidazolinones (Ott et al. (1996) J. Mol. Biol. 263:359-368). Wheat plants produced with some of these rationally designed mutations in the proposed binding sites of the AHAS enzyme have in fact exhibited specific resistance to a single class of herbicides (Ott et al. (1996) J. Mol. Biol. 263:359-368).

Plant resistance to imidazolinone herbicides has also been reported in a number of patents. U.S. Pat. Nos. 4,761,373, 5,331,107, 5,304,732, 6,211,438, 6,211,439 and 6,222,100 generally describe the use of an altered AHAS gene to elicit herbicide resistance in plants, and specifically discloses certain imidazolinone resistant corn lines. U.S. Pat. No. 5,013,659 discloses plants exhibiting herbicide resistance due to mutations in at least one amino acid in one or more conserved regions. The mutations described therein encode either cross-resistance for imidazolinones and sulfonylureas or sulfonylurea-specific resistance, but imidazolinone-specific resistance is not described. Additionally, U.S. Pat. No. 5,731,180 and U.S. Pat. No. 5,767,361 discuss an isolated gene having a single amino acid substitution in a wild-type monocot AHAS amino acid sequence that results in imidazolinone-specific resistance.

In plants, as in all other organisms examined, the AHAS enzyme is comprised of two subunits: a large subunit (catalytic role) and a small subunit (regulatory role) (Duggleby and Pang (2000) J. Biochem. Mol. Biol. 33:1-36). The large subunit (termed AHASL) may be encoded by a single gene as in the case of Arabidopsis and rice or by multiple gene family members as in maize, canola, and cotton. Specific, single-nucleotide substitutions in the large subunit confer upon the enzyme a degree of insensitivity to one or more classes of herbicides (Chang and Duggleby (1998) Biochem J. 333:765-777).

For example, bread wheat, Triticum aestivum L., contains three homoeologous acetohydroxyacid synthase large subunit genes. Each of the genes exhibit significant expression based on herbicide response and biochemical data from mutants in each of the three genes (Ascenzi et al. (2003) International Society of Plant Molecular Biologists Congress, Barcelona, Spain, Ref. No. S10-17). The coding sequences of all three genes share extensive homology at the nucleotide level (WO 03/014357). Through sequencing the AHASL genes from several varieties of Triticum aestivum, the molecular basis of herbicide tolerance in most IMI-tolerant (imidazolinone-tolerant) lines was found to be the mutation S653(At)N (WO 03/014356; WO 03/014357). This mutation is due to a single nucleotide polymorphism (SNP) in the DNA sequence encoding the AHASL protein.

U.S. Pat. No. 5,731,180 discloses the nucleotide and amino acid sequences of a corn AHASL mutant with an amino acid substitution at position 621 of the large subunit which causes imidazolinone-specific resistance. Haughn et al. (Mol. Gen. Genet. 211:266-271, 1988) disclosed the occurrence of imidazolinone resistance in Arabidopsis. Sathasivan et al. (U.S. Pat. No. 5,767,366) identified the imidazolinone-specific resistance in Arabidopsis as being based on a mutation at position 653 in the normal AHASL amino acid sequence. WO 03/014357 discloses partial-length cDNA and amino acid sequences from wheat (Triticum aestivum) corresponding to three wild-type AHASL genes (Als1, Als2 and Als3; also referred to herein below as AHASL1D, AHASL1B, AHASL1A, respectively) as well as imidazolinone-tolerant resistant mutations in Als2, and Als3. To date, nucleotide and amino acid sequences corresponding to the mature wheat AHASL proteins have not been reported.

### SUMMARY OF THE INVENTION

The present invention provides isolated polynucleotide molecules that encode mature, wild-type and herbicide-resistant, wheat (Triticum aestivum L.) AHASL proteins. The polynucleotide molecules of the invention correspond to the three wheat AHASL genes, AHASL1D, AHASL1B, and AHASL1A. The herbicide-resistant AHASL proteins of the invention include, for example, those herbicide-resistant AHASL proteins that possess a substitution in their respective amino acid sequences corresponding to the S653(At)N substitution in the Arabidopsis AHASL protein. The polynucleotide molecules of the invention comprise a nucleotide sequence selected from the group consisting of the nucleotide sequences set forth in SEQ ID NOS: 1, 3, 5, 7, 9, and 11, nucleotide sequences encoding the amino acid sequences set forth in SEQ ID NOS: 2, 4, 6, 8, 10, and 12, and fragments and variants of said nucleotide sequences that encode either a wild-type AHASL protein or a herbicide-resistant AHASL protein, particularly an imidazolinone-resistant AHASL protein having the S653(At)N substitution described supra.

The present invention provides expression cassettes for expressing the polynucleotide molecules of the invention in plants, plant cells, and other, non-human host cells. The expression cassettes comprise a promoter expressible in the plant, plant cell, or other host cells of interest operably linked to a polynucleotide molecule of the invention that encodes a wild-type or imidazolinone-resistant AHASL protein. If desired for expression in plants or plant cells, the expression cassette can also comprise an operably linked chloroplast-targeting sequence that encodes of a chloroplast transit peptide to direct an expressed AHASL protein to the chloroplast. The expression cassettes of the invention find use in a method for enhancing the herbicide tolerance of a plant and a host cell. The method involves transforming the plant or host cell with an expression cassette of the invention, wherein the expression cassette comprises a promoter that is expressible in the plant or host cell of interest and the promoter is operably linked to a polynucleotide of the invention that encodes the imidazolinone-resistant AHASL protein.

The present invention provides transformation vectors comprising a selectable marker gene of the invention. The selectable marker gene comprises a promoter that drives expression in a host cell operably linked to a polynucleotide of the invention. The transformation vector can additionally comprise a gene of interest to be expressed in the host and can also, if desired, include a chloroplast-targeting sequence that is operably linked to the polynucleotide of the invention. Such transformation vectors find use in methods for selecting host cells that are transformed with the gene of interest.

The present invention further provides methods for using the transformation vectors of the invention to select for cells transformed with the gene of interest. Such methods involve the transformation of a host cell with the transformation vector, exposing the cell to levels of an imidazolinone herbicide that would kill or inhibit the growth of a non-transformed host cell, and identifying the transformed host cell by its ability to grow in the presence of the herbicide. In a preferred embodiment of the invention, the host cell is a plant cell and the selectable marker gene comprises a promoter that drives expression in a plant cell.

The present invention provides a method for controlling weeds in the vicinity of a transformed plant of the invention. Such a transformed plant comprises in its genome at least one expression cassette comprising a promoter that drives gene expression in a plant cell, wherein the promoter is operably linked to an herbicide-tolerant AHASL polynucleotide of the invention. The method comprises applying an effective amount of an imidazolinone herbicide to the weeds and to the transformed plant, wherein the transformed plant has increased resistance to the imidazolinone herbicide as compared to an untransformed plant.

The present invention also provides plants, plant tissues, plant cells, seeds, and non-human host cells that are transformed with the at least one polynucleotide, expression cassette, or transformation vector of the invention. Such transformed plants, plant tissues, plant cells, seeds, and non-human host cells have enhanced tolerance or resistance to at least one imidazolinone herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants, including, but not limited to, wheat, rice, maize, corn, sorghum, barely, rye, millet, alfalfa, sunflower, Brassica, soybean, cotton, safflower, peanut, sorghum, millet, tobacco, tomato, and potato.

The present invention provides isolated polypeptides comprising wild-type and imidazolinone-resistant, wheat (Triticum aestivum L.) AHASL proteins. Such isolated imidazolinone-resistant AHASL polypeptides each posses a substitution in their respective amino acid sequences corresponding to the S653(At)N substation in the Arabidopsis AHASL protein. The isolated polypeptides of the invention comprise an amino acid sequence selected from the group consisting of the amino acid sequences set forth in SEQ ID NOS: 2, 4, 6, 8, 10, and 12, the amino acid sequences encoded by nucleotide sequences set forth in SEQ ID NOS: 1, 3, 5, 7, 9, and 11, and fragments and variants of said polypeptides that comprise wild-type or herbicide-tolerant AHAS activity, particularly imidazolinone-tolerant AHAS activity that results from the S653(At)N substitution described supra.

### BRIEF DESCRIPTION OF THE DRAWINGS

FIG. 1 is a table of the percentage sequences identities from pairwise comparisons of the nucleotide and amino acid sequences of wheat AHASL gene coding sequences. Hexaploid refers to sequences from Triticum aestivum. Tetraploid indicates sequences from T. turgidum ssp. durum. Gene 1 corresponds to AHASL1D. Gene 2 corresponds to AHASL1B. Gene 3 corresponds to AHASL1A.

FIG. 2 is a photographic illustration depicting the results of an analysis of the chromosomal location of the three wheat AHASL genes in the Chinese Spring as described in Example 2.

FIG. 3 is a photographic illustration depicting the results of an analysis of the chromosomal location of the three wheat AHASL genes in the Chinese Spring as described in Example 2.

FIG. 4 is a graphical illustration of the correlation between the mutation site and whole plant injury as described in Example 3. 1D, 1B, and 1A denote the wheat AHASL genes, AHASL1D, AHASL1B, and AHASL1A, respectively.

FIG. 5A is a graphical illustration of herbicide-insensitive enzyme activity resulting from the S653(At)N mutation in the AHASL1D, AHASL1B, and AHASL1A proteins of T. aestivum.

FIG. 5B is a graphical illustration of herbicide-insensitive enzyme activity resulting from the S653(At)N mutation in the AHASL1B, and AHASLLA proteins of T. turgidum.

### SEQUENCE LISTING

The nucleotide acid and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleic acid sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence encoding the mature form of a wild-type AHASL1D protein from wheat.

SEQ ID NO: 2 sets forth the amino acid sequence of the mature form of a wild-type AHASL1D protein from wheat.

SEQ ID NO: 3 sets forth the nucleotide sequence encoding the mature form of a wild-type AHASL1B protein from wheat.

SEQ ID NO: 4 sets forth the amino acid sequence of the mature form of a wild-type AHASL1B protein from wheat.

SEQ ID NO: 5 sets forth the nucleotide sequence encoding the mature form of a wild-type AHASL1A protein from wheat.

SEQ ID NO: 6 sets forth the amino acid sequence of the mature form of a wild-type AHASL1A protein from wheat.

SEQ ID NO: 7 sets forth the nucleotide sequence encoding the mature form of a herbicide-tolerant AHASL1 protein from wheat. Relative to SEQ ID NO: 1, SEQ ID NO: 7 includes a C-to-A substitution at nucleotide position 1736.

SEQ ID NO: 8 sets forth the amino acid sequence of the mature form of a herbicide-tolerant AHASL1D protein from wheat. Relative to SEQ ID NO: 2, SEQ ID NO: 8 includes a Ser-to-Asn substitution at amino acid position 579.

SEQ ID NO: 9 sets forth the nucleotide sequence encoding the mature form of a herbicide-tolerant AHASL1B protein from wheat. Relative to SEQ ID NO: 3, SEQ ID NO: 9 includes a C-to-A substitution at nucleotide position 1736.

SEQ ID NO: 10 sets forth the amino acid sequence of the mature form of a herbicide-tolerant AHASL1B protein from wheat. Relative to SEQ ID NO: 4, SEQ ID NO: 10 includes a Ser-to-Asn substitution at amino acid position 579.

SEQ ID NO: 11 sets forth the nucleotide sequence encoding the mature form of a herbicide-tolerant AHASL1A protein from wheat. Relative to SEQ ID NO: 5, SEQ ID NO: 1 includes a C-to-A substitution at nucleotide position 1736.

SEQ ID NO: 12 sets forth the amino acid sequence of the mature form of a herbicide-tolerant AHASL1A protein from wheat. Relative to SEQ ID NO: 6, SEQ ID NO: 12 includes a Ser-to-Asn substitution at amino acid position 579.

### DETAILED DESCRIPTION OF THE INVENTION

The invention is directed to polynucleotide molecules encoding mature, wheat (Triticum aestivum L.) AHASL proteins, particularly polynucleotide molecules encoding wild-type and herbicide-resistant wheat AHASL proteins. Such mature AHASL proteins lack the chloroplast transit peptide that facilitates transport of these proteins into the chloroplast. In particular, the mature, herbicide-resistant wheat AHASL proteins comprise an imidazolinone-resistant AHASL activity. More particularly, the mature, herbicide-resistant wheat AHASL proteins comprise a substitution in their respective amino acid sequences corresponding to the S653(At)N substitution in the Arabidopsis AHASL protein. The polynucleotide molecules of the invention correspond to the three wheat AHASL genes, AHASL1D, AHASL1B, and AHASL1A. The polynucleotide sequences of the invention find use in a method for enhancing the herbicide resistance of plants and host cells. The polynucleotides find further use as selectable marker genes for use in methods for selecting transformed cells, tissues, and organisms, particularly plants and plant cells.

Compositions of the invention include isolated polynucleotide molecules encoding wild-type acetohydroxy acid synthases and isolated polynucleotide molecules encoding herbicide-tolerant or herbicide-resistant acetohydroxy acid synthases that are involved in methods for making plants tolerant or resistant to herbicides at levels that would normally kill, or cease the growth of, a plant. Similarly, by “herbicide-tolerant AHASL protein” or “herbicide-resistant AHASL protein” it is intended that such AHASL proteins display higher AHAS activity, relative to the AHAS activity of a wild-type AHASL, when in the presence of an imidazolinone herbicide at a concentration that is to known to inhibit the AHAS activity of the wild-type AHASL protein. Such herbicide-tolerant or herbicide-resistant AHASL proteins of the invention are encoded by the herbicide-tolerant or herbicide-resistant AHASL polynucleotides.

By “wild-type AHAS activity” is intended to mean the AHAS activity of a wild-type AHASL protein. By “herbicide-tolerant AHAS activity” or “herbicide-resistant AHAS activity” is intended to mean the AHAS activity of a “herbicide-tolerant AHASL protein” or “herbicide-resistant AHASL protein”.

In particular, the present invention provides isolated nucleic acid molecules comprising nucleotide sequences encoding the amino acid sequences shown in SEQ ID NOS: 2, 4, 6, 8, 10, and 12, and fragments and variants thereof. Further provided are polypeptides having an amino acid sequence encoded by a nucleic acid molecule described herein, for example those set forth in SEQ ID NOS: 1, 3, 5, 7, 9, and 11, and fragments and variants thereof.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, preferably culture medium represents less than about 30%, 20%, 10%, 5%, or 1% (by dry weight) of chemical precursors or non-protein-of-interest chemicals.

Fragments and variants of the disclosed nucleotide sequences are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native mature AHASL protein and hence herbicide-tolerant AHAS activity. Alternatively, fragments of a nucleotide sequence that are useful as hybridization probes generally do not encode fragment proteins retain biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of an AHASL nucleotide sequence that encodes a biologically active portion of a wild-type or herbicide-tolerant AHASL protein of the invention will encode at least 15, 25, 30, 50, 100, 150, 200, 250, 350, 400, 450, 500, 525, 550, or 575 contiguous amino acids, or up to the total number of amino acids present in a full-length AHASL protein of the invention (for example, 596 amino acids for each of SEQ ID NOS: 2, 4, 6, 8, 10, and 12, respectively). Fragments of an AHASL protein nucleotide sequence that are useful as hybridization probes or PCR primers generally need not encode a biologically active portion of an AHASL protein.

Thus, a fragment of a AHASL nucleotide sequence may encode a biologically active portion of a AHASL protein, or it may be a fragment that can be used as a hybridization probe or PCR primer using methods disclosed below. A biologically active portion of a AHASL protein can be prepared by isolating a portion of one of the AHASL nucleotide sequences of the invention, expressing the encoded portion of the herbicide-tolerant AHASL protein (e.g., by recombinant expression in vitro), and assessing the activity of the portion of the wild-type or herbicide-tolerant AHASL protein. Nucleic acid molecules that are fragments of a wild-type or herbicide-tolerant AHASL nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,650, 1,700, or 1,750 nucleotides, or up to the number of nucleotides present in a full-length a herbicide-tolerant AHASL nucleotide sequence disclosed herein (for example, 1788 nucleotides for each of SEQ ID NOS: 1, 3, 5, 7, 9, and 12, respectively).

By “variants” is intended substantially similar sequences. For nucleotide sequences, conservative variants include those sequences that, because of the degeneracy of the genetic code, encode the amino acid sequence of one of AHASL polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an AHASL protein of the invention. Generally, variants of a particular nucleotide sequence of the invention will have at least about 70%, 75%, generally at least about 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

Variants of a particular nucleotide sequence of the invention (i.e., the reference nucleotide sequence) can also be evaluated by comparison of the percent sequence identity between the polypeptide encoded by a variant nucleotide sequence and the polypeptide encoded by the reference nucleotide sequence. Thus, for example, isolated nucleic acids that encode a polypeptide with a given percent sequence identity to the polypeptide of SEQ ID NO: 2 are disclosed. Percent sequence identity between any two polypeptides can be calculated using sequence alignment programs described elsewhere herein using default parameters. Where any given pair of nucleotide sequences of the invention is evaluated by comparison of the percent sequence identity shared by the two polypeptides they encode, the percent sequence identity between the two encoded polypeptides is at least about 40%, 45%, 50%, 55%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, wild-type or herbicide-tolerant AHAS activity as described herein. Such variants may result from, for example, genetic polymorphism or from human manipulation. Biologically active variants of a native herbicide-tolerant AHASL protein of the invention will have at least about 40%, 45%, 50%, 55%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the herbicide-tolerant AHASL protein can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

The herbicide-resistant AHASL proteins of the invention include, but are not limited to, the proteins comprising the amino acid sequences set forth in SEQ ID NOS: 8, 10, and 12. Each of these amino acid sequences, comprises an amino acid substitution (relative to their respective wild-type sequences,) that corresponds to the S653(At)N substitution described above. For SEQ ID NOS: 8, 10, and 12, this substitution is at amino acid residue or position 579. The herbicide-resistant wheat AHASL proteins of the invention also encompass proteins comprising variants and fragments of the amino acid sequences set forth in SEQ ID NOS: 8, 10, and 12, and which also comprise an asparagine at amino acid position 579 or equivalent position and herbicide-tolerant AHAS activity. By “equivalent position” is intended to mean a position in an AHASL protein that is equivalent to amino acid position or residue 653 in the imidazolinone-resistant Arabidopsis AHASL protein disclosed in U.S. Pat. No. 5,767,366 or amino acid position 579 in SEQ ID NOS: 2, 4, 6, 8, 10, and 12 of the present invention. Preferably, the substitution of asparagine for a serine residue at such an equivalent position of an AHASL protein can result in an AHASL protein comprising herbicide-tolerant AHAS activity. Additionally, the present invention encompasses the polynucleotide molecules encoding such herbicide-resistant wheat AHASL proteins.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired wild-type or herbicide-tolerant AHASL activity. Obviously, the mutations that will be made in the DNA sequence encoding the variant must not place the sequence out of reading frame and preferably will not create complementary regions that could produce secondary mRNA structure. See, EP Patent Application Publication No. 75,444.

In addition, the herbicide-resistant AHASL proteins of the invention include, but are not limited to, herbicide-resistant AHASL proteins comprising the S653(At)N substitution described above and/or at least one other mutation that is known to confer herbicide resistance on an AHASL protein. See, WO 03/013255, WO 03/014356, WO 03/014357, and U.S. Provisional Patent Application Ser. No. 60/473,828; each of which is herein incorporated by reference. In an embodiment of the invention, the herbicide-resistant AHASL proteins of the invention can comprise one, two, three, or more of such mutations. The present invention further encompasses the polynucleotides molecules that encode such herbicide-resistant AHASL proteins.

Thus, the herbicide-resistant AHASL proteins of the invention are not limited to those AHASL proteins that comprise the S653(At)N substitution described above. In particular, the present invention additionally encompasses herbicide-resistant variants and fragments of the AHASL proteins comprising the amino acid sequences set forth in SEQ ID NOS: 2, 4, and 6, and herbicide-resistant variants and fragments of the proteins encoded by the nucleotide sequences set forth in SEQ ID NOS: 1, 3, and 5. Such herbicide-resistant variants and fragments of the AHASL proteins can be produced, for example, by altering the nucleotide sequence encoding AHASL proteins of the inventions as described herein to include one or more the mutations that are known to confer herbicide resistance on the AHASL proteins encoded thereby. Such mutations are described above. The present invention further encompasses the polynucleotides molecules that encode such herbicide-resistant variants and fragments.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, AHASL function can be evaluated by AHAS enzyme activity assays in the presence and absence of an imidazolinone herbicide. See, for example, Singh et al. ((1988) Anal. Biochem. 171:173-179), herein incorporated by reference.

Variant nucleotide sequences and proteins also encompass sequences and proteins derived from a mutagenic and recombinogenic procedure such as DNA shuffling. With such a procedure, one or more different herbicide-tolerant AHASL protein sequences can be manipulated to create a new herbicide-tolerant AHASL protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the herbicide-tolerant AHASL gene of the invention and other known AHASL genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire AHASL sequences set forth herein or to fragments thereof are encompassed by the present invention. Thus, isolated sequences that encode for a herbicide-tolerant AHASL protein and which hybridize under stringent conditions to the AHASL sequence disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the herbicide-tolerant AHASL sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire AHASL polynucleotide disclosed herein, or one or more portions thereof, may be used as a probe capable of specifically hybridizing to corresponding AHASL polynucleotide and messenger RNAs. To achieve specific hybridization under a variety of conditions, such probes include sequences that are unique among AHASL polynucleotides and are preferably at least about 10 nucleotides in length, and most preferably at least about 20 nucleotides in length. Such probes may be used to amplify corresponding AHASL polynucleotides from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 110° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches. For the present invention, unless otherwise stated herein, for comparisons of a nucleotide or amino acid sequence of the present invention to another sequence, the comparison window is the length of the full-length sequence of the invention.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained for alignment of the full-length sequences of the invention to other sequences using Vector NTI Version 7.1 (Informax, Inc., Frederick, Md., USA) using default parameters. Vector NTI Version 7.1 uses the Clustal W algorithm to generate multiple sequence alignments. By “equivalent program” is intended any sequence comparison program that, for any two sequences in question, generates an alignment having identical nucleotide or amino acid residue matches and an identical percent sequence identity when compared to the corresponding alignment generated by Vector NTI Version 7.1.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i)The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The AHASL polynucleotides of the invention are provided in expression cassettes for expression in the plant of interest. The cassette will include 5′ and 3′ regulatory sequences operably linked to a AHASL sequence of the invention. By “operably linked” with respect to a promoter is intended a functional linkage between a promoter and a second sequence, wherein the promoter sequence initiates and mediates transcription of the DNA sequence corresponding to the second sequence. Generally, “operably linked” means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the AHASL polynucleotide to be under the transcriptional regulation of the regulatory regions. The expression cassette may additionally contain selectable marker genes.

The polynucleotide molecules of the invention include, for example, polynucleotide molecules comprising the nucleotide sequences set forth in SEQ ID NOS: 1, 3, 5, 7, 9, and 11. It is recognized that such nucleotide sequences do not include an initiation codon. If desired for expression in a host cell or plant, an initiation codon, such as an ATG, can be operably linked to the nucleotide sequence of the invention. Alternatively, if chloroplast expression is desired, a chloroplast-targeting sequence comprising such an initiation codon can be operably linked to a nucleotide sequence of the invention.

The expression cassette will include in the 5′-3′ direction of transcription, a transcriptional and translational initiation region (i.e., a promoter), a AHASL polynucleotide sequence of the invention, and a transcriptional and translational termination region (i.e., termination region) functional in plants. The promoter may be native or analogous, or foreign or heterologous, to the plant host and/or to the AHASL polynucleotide sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the AHASL polynucleotide sequence of the invention, it is intended that the promoter is not the native or naturally occurring promoter for the operably linked AHASL polynucleotide sequence of the invention. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of the AHASL protein of the invention or in the plant or plant cell, or confer a herbicide-tolerance phenotype on the plant or plant cell. Thus, the phenotype of the plant or plant cell is altered.

The termination region may be native with the transcriptional initiation region, may be native with the operably linked AHASL polynucleotide sequence of interest, may be native with the plant host, or may be derived from another source (i.e., foreign or heterologous to the promoter, the AHASL polynucleotide of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92: 1-1 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced AHASL expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505.

In one embodiment, the polynucleotide molecules of the invention are targeted to the chloroplast for expression. In this manner, where the polynucleotide molecule of interest is not directly inserted into the chloroplast, the expression cassette will additionally contain an operably linked nucleic acid sequence encoding a transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the AHASL polynucleotide of the invention such that the two sequences are contiguous and in the same reading frame. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast-targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl) shikimate-3-phosphate synthase (EPSPS) (Archer et al. (1990) J Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. Alternatively, the chloroplast targeting sequence for a wheat AHASL gene can be isolated and operably linked to an AHASL nucleotide molecule of the invention.

The polynucleotide molecules of the invention can be used to transform the chloroplast genome of a plant. Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The AHASL polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See, for example, U.S. Pat. No. 5,380,831, herein incorporated by reference.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

It is recognized that with these nucleotide sequences, antisense constructions, complementary to at least a portion of the messenger RNA (mRNA) for the AHASL polynucleotide molecule can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

The AHASL polynucleotides of the invention can be used in method for enhancing the tolerance of a plant, plant cell, or other host cell to pyrimidyloxybenzoate, pyrimidylthiobenzoate, and imidazolinone herbicides. The AHASL polynucleotides of the invention can also be used in methods for selecting transformed plants, plant cells, and, other host cells that involve exposing the plants, plant cells, and host cells to imidazolinone herbicides. For the present invention, the imidazolinone herbicides include, but are not limited to, PURSUIT® (imazethapyr), CADRE® (imazapic), RAPTOR® (imazamox), SCEPTER® (imazaquin), ASSERT® (imazethabenz), ARSENAL® (imazapyr), a derivative of any of the aforementioned herbicides, or a mixture of two or more of the aforementioned herbicides, for example, imazapyr/imazamox (ODYSSEY®). More specifically, the imidazolinone herbicide can be selected from, but is not limited to, 2-(4-isopropyl-4-methyl-5-oxo-2-imidiazolin-2-yl)-nicotinic acid, [2-(4-isopropyl)-4-][methyl-5-oxo-2-imidazolin-2-yl)-3-quinolinecarboxylic]acid, [5-ethyl-2-(4-isopropyl-]4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, [2-(4-isopropyl-4-methyl-5-oxo-2-]imidazolin-2-yl)-5-methylnicotinic acid, and a mixture of methyl [6-(4-isopropyl-4-]methyl-5-oxo-2-imidazolin-2-yl)-m-toluate and methyl [2-(4-isopropyl-4-methyl-5-]oxo-2-imidazolin-2-yl)-p-toluate. The use of 5-ethyl-2-(4-isopropyl-4-methyl-5-oxo-2-imidazolin-2-yl)-nicotinic acid and [2-(4-isopropyl-4-methyl-5-oxo-2-imidazolin-2-]yl)-5-(methoxymethyl)-nicotinic acid is preferred. the use of [2-(4-isopropyl-4-]methyl-5-oxo-2-imidazolin-2-yl)-5-(methoxymethyl)-nicotinic acid is particularly preferred. For the present invention, the pyrimidylthiobenzoate herbicides include, but are not limited to, STAPLE® (pyrithiobac sodium).

The methods of the invention involve exposing transformed plants, transformed plant cells, and transformed host cells to a herbicide, particularly a pyrimidyloxybenzoate, pyrimidylthiobenzoate, or imidazolinone herbicide, more particularly an imidazolinone herbicide. The preferred amount or concentration of the herbicide is an “effective amount” or “effective concentration.” By “effective amount” and “effective concentration” is intended an amount and concentration, respectively, that is sufficient to kill or inhibit the growth of a similar, untransformed, plant, plant cell, or host cell, but said amount does not kill or inhibit as severely the grow of the transformed plants, transformed plant cells,, and transformed host cells. By “similar, untransformed, plant, plant cell or host cell” is intended a plant, plant, plant tissue, plant cell, or host cell, respectively, that lacks the particular polynucleotide of the invention that was used to making the transformed plant, transformed plant cell, of transformed host cell of the invention. The use of the term “untransformed” is not, therefore, intended to imply that a plant, plant cell, or other host cell lacks recombinant DNA in its genome.

The present invention may be used for transformation of any plant species, including, but not limited to, monocots and dicots. Examples of plant species of interest include, but are not limited to, wheat (Triticum aestivum, Triticum turgidum ssp. durum), corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinusponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, wheat, corn, alfalfa, sunflower, Brassica, soybean, cotton, safflower, peanut, sorghum, millet, tobacco, etc.), more preferably grain plants (for example, wheat, rice, corn, barley, sorghum, rye, millet, etc.), most preferably wheat plants.

The present invention also encompasses non-transgenic plants, particularly non-transgenic wheat plants, comprising in their genomes one or more of the herbicide resistant AHASL polynucleotides of the invention. Such wheat plants are herbicide resistant and can be produced from wild-type wheat plants via any mutagenesis method that is known in the art. See, for example, U.S. Pat. No. 6,339,184; herein incorporated by reference. The present invention additionally encompasses plant cells, plant parts, plant tissues, seed and the progeny of such herbicide-resistant plants.

The host cells of the invention include prokaryotic and eukaryotic cells, particularly bacterial cells, fungal cells, and animal cells. Such fungal cells include, but are not limited to, yeast cells, and such animal cells, include but are not limited to, insect cells and mammalian cells.

While the AHASL polynucleotides of the invention find use as selectable marker genes for plant transformation of the invention, the expression cassettes of the invention can include another selectable marker gene for the selection of transformed cells. Selectable marker genes, including those of the present invention, are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10: 143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene, including the selectable marker genes of the invention, can be used in the present invention.

The transformation vectors of the invention can be used to produce plants transformed with a gene of interest. The transformation vector will comprise a selectable marker gene of the invention and a gene of interest to be introduced and typically expressed in the transformed plant.

The genes of interest vary depending on the desired outcome. For example, various changes in phenotype can be of interest including modifying the fatty acid composition in a plant, altering the amino acid content of a plant, altering a plant's insect and/or pathogen defense mechanisms, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in various seed components, for example, oil, starch, protein, and soluble sugars, and those which favorably affect agronomic performance such as insect and disease resistance, and tolerance to environmental stresses such as, for example, cold, heat, and drought.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The use herein of the terms “polynucleotides”, “polynucleotide molecules”, “nucleotide molecules”, “nucleotide constructs” and the like is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral polynucleotides. Generally, such methods involve incorporating a nucleotide construct of the invention within a viral DNA or RNA molecule. It is recognized that the an imidazolinone-tolerant AHASL protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

Additionally provided herein is a method for controlling weeds in the vicinity of a plant transformed with at least one AHASL polynucleotide of the invention. The method comprises applying an effective amount of a herbicide, particularly an effective amount of an imidazolinone herbicide, to the weeds and to the transformed plant or to the soil in which the weeds and the transformed plant occur, wherein the plant has increased resistance to the herbicide as compared to an untransformed plant. By “effective amount of an herbicide” is intended an amount that is sufficient to kill or retard the growth of the desired weeds in the vicinity of the transformed plant and is also sufficient to kill a untransformed plant that is same as the transformed plant but lacks in its genome at least one herbicide-tolerant AHASL polynucleotide of the invention. In addition, an effective amount of an herbicide does not kill the transformed plant of the invention when applied to the transformed plant, and preferably, does not significantly retard the growth of, or significantly injure, the transformed plant. Typically, the effective amount of an herbicide is an amount that is routinely used in agricultural production systems to kill weeds. Such an amount is known to those of ordinary skill in the art.

In such a method for controlling weeds, the transformed plants are preferably crop plants, including, but not limited to, wheat, rice, maize, corn, sorghum, barely, rye, millet, alfalfa, sunflower, Brassica, soybean, cotton, safflower, peanut, sorghum, millet, tobacco, tomato, and potato.

By providing plants having increased resistance to herbicides, particularly imidazolinone herbicides, a wide variety of formulations can be employed for protecting plants from weeds, so as to enhance plant growth and reduce competition for nutrients. A herbicide can be used by itself for pre-emergence, post-emergence, pre-planting and at planting control of weeds in areas surrounding the plants described herein or an imidazolinone herbicide formulation can be used that contains other additives. The herbicide can also be used as a seed treatment. Additives found in an herbicide formulation include other herbicides, detergents, adjuvants, spreading agents, sticking agents, stabilizing agents, or the like. The herbicide formulation can be a wet or dry preparation and can include, but is not limited to, flowable powders, emulsifiable concentrates and liquid concentrates. The herbicide and herbicide formulations can be applied in accordance with conventional methods, for example, by spraying, irrigation, dusting, or the like.

### EXAMPLE 1

### The AHASL Large Subunit is Encoded by Three Genes

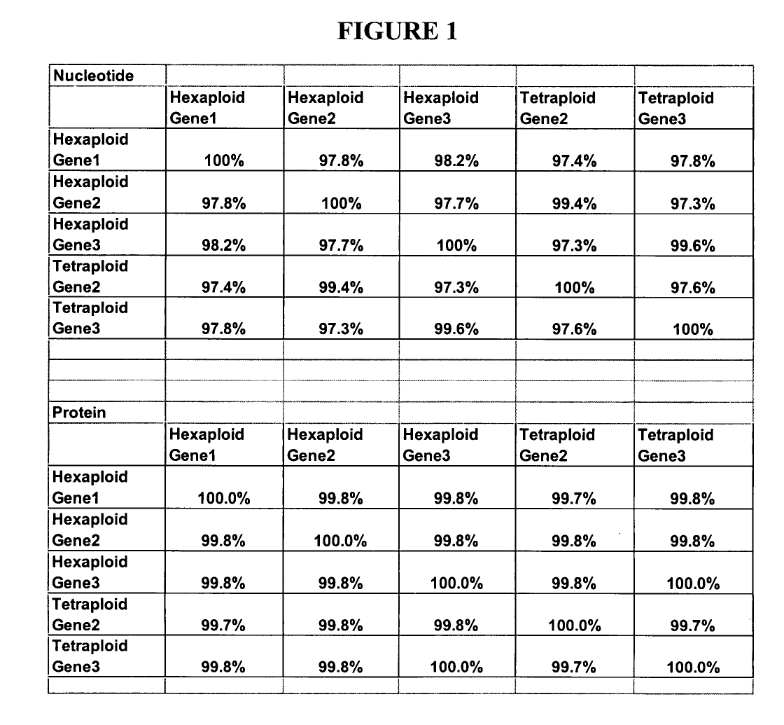

In the course of a wheat mutagenesis program, thousands of independently derived lines were analyzed by herbicide spray assays. To understand the molecular basis of tolerance, an effort was made to identify the active genes in wheat. Cloning of AHASL genes from wild-type and imidazolinone-resistant wheat plants was accomplished by designing degenerate PCR primers based on previously cloned AHASL nucleotide molecules. Cloned PCR products fell into three closely related groups, suggesting that there are three AHASL genes in hexaploid wheat. This is consistent with each of the diploid genomes possessing a single AHASL gene. Subsequent analysis of EST data indicated that there are only three active copies of AHASL. In addition, only three independently segregating resistance genes have been found (Pozniak and Hucl, in press). The nearly full-length sequences of wheat AHASL genes have been determined by RACE-PCR. The nucleotide and amino acid sequences (SEQ ID NOS: 1-12) that are set forth in the sequence listing are from the wheat (Triticum aestivum L.) variety ‘Gunner’. Elucidation of the complete transcribed sequence was hampered by the very high GC content of the 5′ portion of the coding sequence. Each of the three genes are approximately 98% identical along their 1788 bp length and the encoded proteins differ from each other only by one amino acid (FIG. 1). The closely related tetraploid species, Triticum turgidum L. (durum wheat) contains two genes encoding proteins identical (gene 3) or differing by one amino acid (gene 2) compared to their cognates in hexaploid wheat.

### EXAMPLE 2

### Wheat AHASL Genes Map to the Long Arm of Chromosome 6

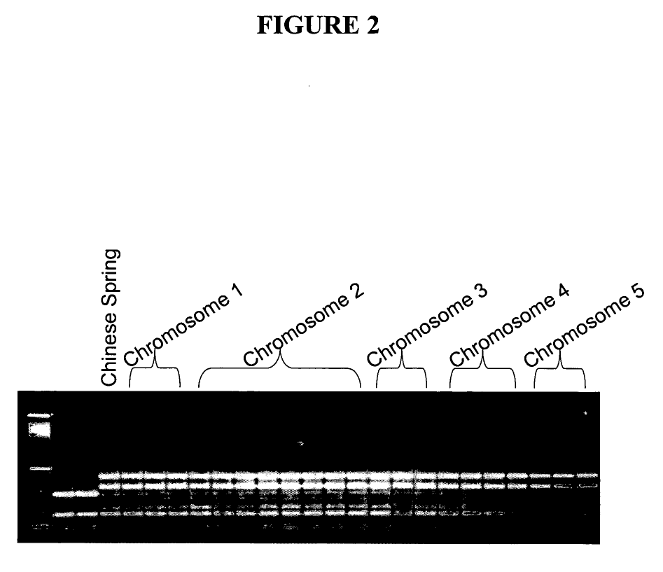

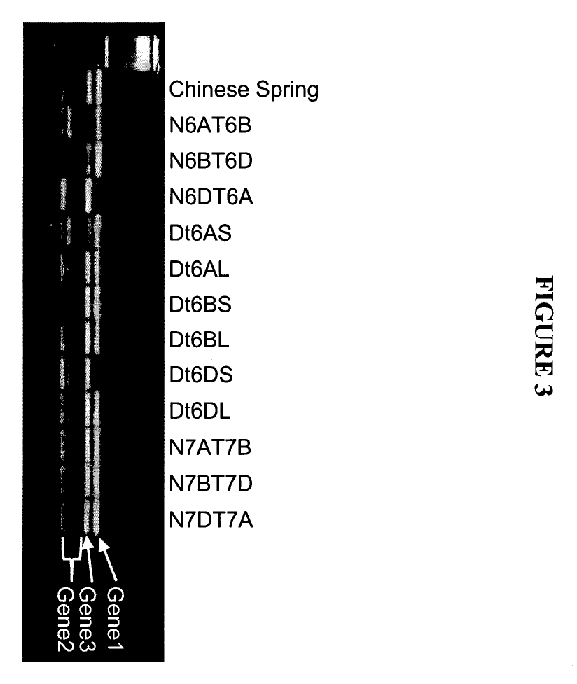

In order to determine the chromosomal location of the three genes, a collection of aneuploid stocks of “Chinese Spring” was used in conjunction with gene-specific, cleaved-amplified-polymorphism (CAPS) markers (Pozniak et al. submitted). Gene 1 was found to be missing from N6DT6A and Dt6DS, while gene 2 was not present in lines N6BT6D and Dt6BS, and gene 3 was absent in lines N6AT6B and Dt6AS (FIGS. 2-3). This indicates that gene 1 is located on the long arm of chromosome 6D, gene 2 on 6B and gene 3 on 6A. The homoeologous genes, genes 1-3, are now renamed AHASL1D, AHALSL1B, and AHASL1A, respectively, with the last letter indicating the genome.

### EXAMPLE 3

### The Level of Tolerance is Influenced by the AHASL Gene that is Mutated

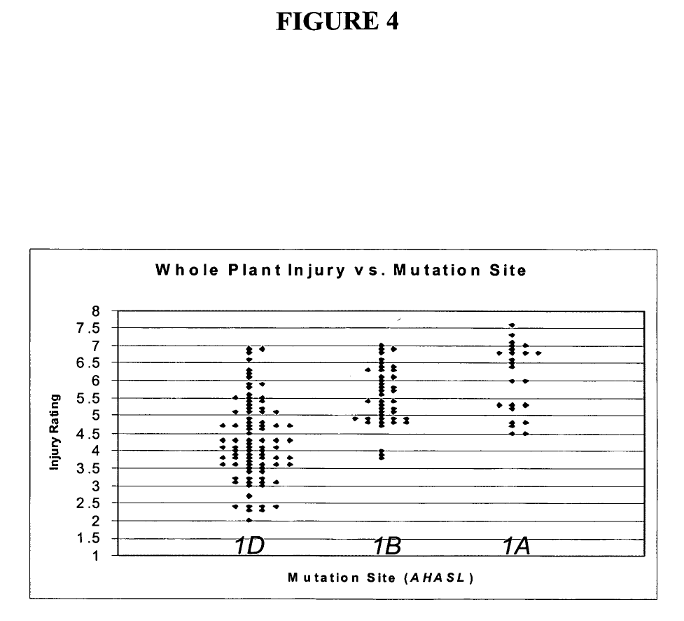

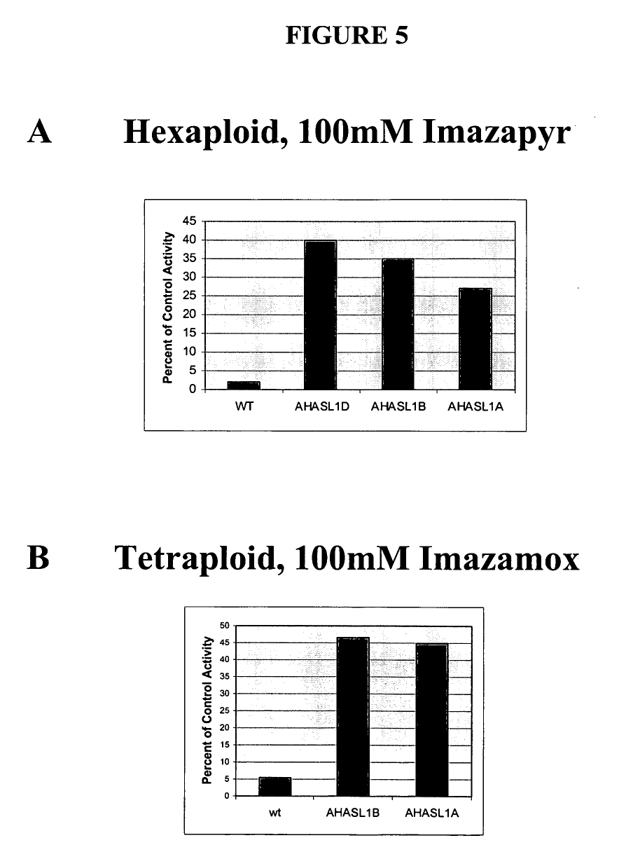

The most common mutation observed in wheat results in a Ser to Asn substitution at the position equivalent to Ser653 in Arabidopsis (termed S653(At)N). Typically, 24 individuals from each mutagenized line were scored on a 0-9 rating scale (0=no injury; 9=highest injury) after spraying with a rate of imazamox sufficient for detecting differences in the greenhouse-grown plants (FIG. 4). For each line, the specific gene mutated was determined. A clear correlation was found between the ratings from herbicide spray assays and the specific homoeolog that was mutated at this position. Mutations in AHASL1D resulted in higher tolerance (median injury=4.0) as compared to 1B (5.4) and 1A (6.4).

### EXAMPLE 4

### Each of the AHASL Genes in Hexaploid Wheat Contribute Varying Amounts of Activity to the Enzyme Pool

To obtain an understanding of the effect of each mutation on the level of enzyme sensitivity to the herbicides, AHAS assays were performed on S653(At)N mutants in all three genes using individuals from several backgrounds (representative data in FIG. 6A). The results parallel the spray assay data in that AHASL1D conferred the highest level of insensitivity (38%), while AHASL1A and 1B showed lower levels of activity (33% and 25% respectively) in the presence of 100 μM imazapyr. A comparison of extracts from mutants in the CDC Teal background showed a similar relationship with AHAS1D having 40% activity in the presence of 100 μM imazethapyr and AHAS1B containing 30% activity. Also, a double mutant between 1A and 1D retained 63% of AHAS activity (Pozniak et al. submitted). Taken together, the data suggest that AHASL1D contributes the greatest amount of activity to the enzyme pool and that the level of resistance is roughly additive. In durum wheat (T. turgidum) each homeolog appears to contribute equally to the AHAS pool (FIG. 6B).

### EXAMPLE 5

### Herbicide-Resistant Wheat AHASL Proteins

The present invention discloses both the nucleotide and amino acid sequences for mature, wild-type wheat AHASL polypeptides and for mature, herbicide-resistant wheat AHASL polypeptides. Plants comprising herbicide-resistant AHASL polypeptides have been previously identified, and a number of conserved regions of AHASL polypeptides that are the sites of amino acids substitutions that confer herbicide resistance have been described. See, Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam; and Devine and Shukla, (2000) Crop Protection 19:881-889.

Using the AHASL sequences of the invention (SEQ ID NOS: 1, 3, 5, 7, 9, and 11) and methods known to those of ordinary skill in art and disclosed herein, one can produce additional polynucleotides encoding herbicide-resistant AHASL polypeptides having one, two, three, or more amino acid substitutions at the identified sites in these conserved regions. Table 6 provides the conserved regions of AHASL proteins, the amino acid substitutions known to confer herbicide resistance within these conserved regions, and the corresponding amino acids in the wheat AHASL proteins set forth in SEQ ID NOS: 2, 4, 6, 8, 10, and 12.

TABLE 1
Amino Acid Substitutions in Conserved Regions of AHASL Polypeptides
that are Known to Confer Herbicide-Resistance

| | | | Amino acid position |
|---|---|---|---|
| Conserved region¹ | Mutation² | Reference | in wheat |
| VFAYPGGASMEIHQALTRS³ | Ala₁₂₂ to Thr | Bernasconi et al.⁴ | Ala₄₈ |
| | | Wright & Penner⁵ | |
| AITGQVPRRMIGT³ | Pro₁₉₇ to Ala | Boutsalis et al.⁶ | Pro₁₂₃ |
| | Pro₁₉₇ to Thr | Guttieri et al.⁷ | |
| | Pro₁₉₇ to His | Guttieri et al.⁸ | |
| | Pro₁₉₇ to Leu | Guttieri et al.⁷ | |
| | | Kolkman et al.⁹ | |
| | Pro₁₉₇ to Arg | Guttieri et al.⁷ | |
| | Pro₁₉₇ to Ile | Boutsalis et al.⁶ | |
| | Pro₁₉₇ to Gln | Guttieri et al.⁷ | |
| | Pro₁₉₇ to Ser | Guttieri et al.⁷ | |
| AFQETP³ | Ala₂₀₅ to Asp | Hartnettef al.¹⁰ | Ala₁₃₁ |
| | | Simpson¹¹ | |
| | Ala₂₀₅ to Val¹¹ | Kolkman et al.⁹ | |
| | | White etal.¹² | |
| QWED³ | Trp₅₇₄ to Leu | Bruniard¹³ | Trp₅₀₀ |
| | | Boutsalis et al.⁶ | |
| IPSGG³ | Ser₆₅₃ to Asn | Devine & | Ser₅₇₉¹⁷ |
| | | Eberlein¹⁴ | |
| | | Lee et al.¹⁵ | |
| | Ser₆₅₃ to Thr | Chang & | |
| | Ser₆₅₃ to Phe | Duggleby¹⁶ | |
| ¹Conserved regions from Devine and Eberlein (1997) “Physiological, biochemical and molecular aspects of herbicide resistance based on altered target sites”. In: Herbicide Activity: Toxicology, Biochemistry and Molecular Biology, | | | |
| # Roe et al. (eds.), pp. 159-185, IOS Press, Amsterdam and Devine and Shukla, (2000) Crop Protection 19:881-889. | | | |
| ²Amino acid numbering corresponds to the amino acid sequence of the Arabidopsis thaliana AHASL polypeptide. | | | |
| ³Each of the amino acid sequences of the wheat AHASL proteins of the invention (SEQ ID NO: 2, 4, 6, 8, 10, and 12) comprise the same conserved region. | | | |
| ⁴Bemasconi et al. (1995) J. Biol. Chem. 270(29):17381-17385. | | | |
| ⁵Wright and Penner (1998) Theor. Appl. Genet. 96:612-620. | | | |
| ⁶Boutsalis et al. (1999) Pestic. Sci. 55:507-516. | | | |
| ⁷Guttieri et al. (1995) Weed Sci. 43:143-178. | | | |
| ⁸Guttieri et al. (1992) Weed Sci. 40:670-678. | | | |
| ⁹Kolkman et al. (2004) Theor. Appl. Genet. 109: 1147-1159. | | | |
| ¹⁰Hartnett et al. (1990) “Herbicide-resistant plants carrying mutated acetolactate synthase genes,” In: Managing Resistance to Agrochemicals: Fundamental Research to Practical Strategies, Green et al. (eds.), American Chemical Soc. Symp., Series No. 421, Washington, D.C., USA | | | |
| ¹¹Simpson (1998) Down to Earth 53(1):26-35. | | | |
| ¹²White et al. (2003) Weed Sci. 51:845-853. | | | |
| ¹³Bruniard (2001) Inheritance of imidazolinone resistance, characterization of cross-resistance pattern, and identification of molecular markers in sunflower (Helianthus annuus L.). Ph.D. Thesis, North Dakota State University, Fargo, ND, USA, pp 1-78. | | | |
| ¹⁴Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam. | | | |
| ¹⁵Lee et al. (1999) FEBS Lett. 452:341-345. | | | |
| ¹⁶Chang and Duggleby (1998) Biochem J. 333:765-777. | | | |
| ¹⁷The present invention discloses SEQ ID NOS: 8, 10, and 12, which set forth the amino acid sequences of mature, herbicide-resistant wheat AHASL1D, AHASL1B, and AHASL1A proteins of the present invention, respectively. Each of these amino acid sequences comprises an Asn at amino acid position 579. The present invention further discloses SEQ ID NOS: 7, 9, and 11, | | | |
| # which set forth polynucleotide sequences encoding the mature herbicide resistant mature, herbicide-resistant wheat proteins. | | | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polynucleotide molecule comprising a nucleotide sequence selected from the group consisting of:
(a) the nucleotide sequence set forth in SEQ ID NO: 1, 3, 5, 7, 9, or 11;
(b) a nucleotide sequence encoding the amino acid sequence set forth in SEQ ID NO: 2, 4, 6, 8, 10, or 12;
(c) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a protein comprising acetohydroxyacid synthase (AHAS) activity;
(d) a nucleotide sequence encoding an amino acid sequence having at least 95% amino acid sequence identity to at least one amino acid sequence selected from the group consisting of SEQ ID NOS: 2, 4, 6, 8, 10, and 12, wherein said nucleotide sequence encodes a protein comprising acetohydroxyacid synthase activity;
(e) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a mature herbicide-tolerant acetohydroxyacid synthase large subunit (AHASL) protein comprising an asparagine at amino acid position 579 or equivalent position;
(f) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a protein comprising herbicide-tolerant AHAS activity; and
(g) a nucleotide sequence that is the complement of at least one of the nucleotide sequences of (a)-(f).

2. The polynucleotide molecule of claim 1, wherein said nucleotide sequence is selected from the group consisting of:
(i) the nucleotide sequence set forth in SEQ ID NO: 7, 9, or 11;
(ii) a nucleotide sequence encoding the amino acid sequence set forth in SEQ ID NO: 8, 10, or 12;
(iii) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a mature herbicide-tolerant AHASL protein comprising an asparagine at amino acid position 579 or equivalent position; and
(iv) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a protein comprising herbicide-tolerant AHAS activity.

3. An expression cassette comprising a promoter expressible in a host cell, said promoter operably linked to the polynucleotide molecule of claim 1.

4. The expression cassette of claim 3, wherein said promoter is expressible in at least one host cell selected from the group consisting of a plant cell, a bacterial cell, an animal cell, and a fungal cell.

5. A transformation vector comprising a gene of interest and a selectable marker gene, said selectable marker gene comprising a promoter operably linked to the polynucleotide molecule of claim 2.

6. A transformed plant comprising stably incorporated in its genome at least one expression cassette comprising a polynucleotide molecule operably linked to a promoter that drives expression in a plant cell, wherein said polynucleotide molecule comprises a nucleotide sequence selected from the group consisting of:
(a) the nucleotide sequence set forth in SEQ ID NO: 1, 3, 5, 7, 9, or 11;
(b) a nucleotide sequence encoding the amino acid sequence set forth in SEQ ID NO: 2, 4, 6, 8, 10, or 12;
(c) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a protein comprising AHAS activity;
(d) a nucleotide sequence encoding an amino acid sequence having at least 95% amino acid sequence identity to at least one amino acid sequence selected from the group consisting of SEQ ID NOS: 2, 4, 6, 8, 10, and 12, wherein said nucleotide sequence encodes a protein comprising acetohydroxyacid synthase activity;
(e) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a mature herbicide-tolerant AHASL protein comprising an asparagine at amino acid position 579 or equivalent position;
(f) a nucleotide sequence having at least 95% nucleotide sequence identity to the complement of at least one nucleotide sequence selected from the group consisting of SEQ ID NOS: 1, 3, 5, 7, 9, and 11, wherein said nucleotide sequence encodes a protein comprising herbicide-tolerant AHAS activity; and
(g) a nucleotide sequence that is the complement of at least one of the nucleotide sequences of (a)-(f).

7. The transformed plant of claim 6, wherein said expression cassette further comprises an operably linked chloroplast-targeting sequence.

8. The transformed plant of claim 6, wherein said plant comprises in its genome at least one non-transgenic, imidazolinone-tolerant AHASL gene.

9. The transformed plant of claim 6, wherein said plant is a monocot or a dicot.

10. The transformed plant of claim 9, wherein said monocot is selected from the group consisting of wheat, triticale, maize, rice, sorghum, rye, millet, and barley.

11. The transformed plant of claim 9, wherein said dicot is selected from the group consisting of alfalfa, sunflower, Brassica, soybean, cotton, safflower, peanut, tobacco, tomato, and potato.

12. The transformed plant of claim 6, wherein said plant has enhanced resistance to at least one herbicide, relative to an untransformed plant.

13. A transformed seed of the plant of claim 6, wherein said seed comprises said expression cassette.

14. A transformed plant cell comprising stably incorporated in its genome at least one expression cassette comprising the polynucleotide molecule of claim 1 operably linked to a promoter that drives expression in a plant cell.

15. A method for enhancing the herbicide-resistance of a plant comprising the steps of:
transforming at least one cell of said plant with at least one expression cassette comprising the polynucleotide molecule of claim 2 operably linked to a promoter that drives expression in a plant cell; and
regenerating a stably transformed plant from said cell, wherein said transformed plant has enhanced resistance to at least one herbicide relative to an untransformed plant.

16. The method of claim 15, wherein said expression cassette further comprises an operably linked chloroplast-targeting sequence.

17. The method of claim 15, wherein said herbicide is selected from the group consisting of an imidazolinone herbicide, a pyrimidyloxybenzoate herbicide, and a pyrimidylthiobenzoate herbicide.

18. The method of claim 17, wherein said imidazolinone herbicide is selected from the group consisting of: [2-(4-isopropyl-4-methyl-5-oxo-2-]imidiazolin-2-yl)-nicotinic acid, 2-(4-isopropyl)-4-methyl-5-oxo-2-imidazolin-2-yl)-3-quinolinecarboxylic acid, [5-ethyl-2-(4-isopropyl-4-methyl-]5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, a mixture of methyl 6-(4-isopropyl-4-methyl-5-oxo-2-imidazolin-2-yl)-m-toluate and methyl [2-(4-]isopropyl-4-methyl-5-oxo-2-imidazolin-2-yl)-p-toluate, and a mixture thereof.

19. A method for selecting a transformed plant cell comprising the steps of:
transforming a plant cell with a plant transformation vector comprising a selectable marker gene, said selectable marker gene comprising the polynucleotide molecule of claim 2 operably linked to a promoter that drives expression in a plant cell;
exposing said transformed plant cell to a herbicide at a concentration that inhibits the growth of an untransformed plant cell; and
identifying said transformed plant cell by its ability to grow in the presence of said herbicide.

20. The method of claim 19, wherein said selectable marker gene further comprises an operably linked chloroplast-targeting sequence.

21. The method of claim 19, wherein said plant transformation vector further comprises at least one gene of interest.

22. A method of controlling weeds in the vicinity of a transformed plant, said method comprising applying an effective amount of an herbicide to the weeds and to the transformed plant, wherein said transformed plant has increased resistance to the herbicide as compared to an untransformed plant, and the transformed plant comprises in its genome at least one expression cassette comprising the polynucleotide molecule of claim 2 operably linked to a promoter that drives expression in a plant cell.

23. The method of claim 22, wherein said selectable marker gene further comprises an operably linked chloroplast-targeting sequence.

24. The method of claim 22, wherein said herbicide is selected from the group consisting of an imidazolinone herbicide, a pyrimidyloxybenzoate herbicide, and a pyrimidylthiobenzoate herbicide.

25. A non-human host cell comprising the expression cassette of claim 3.

26. A non-human host cell comprising the transformation vector of claim 5.

27. An isolated polypeptide comprising an amino acid sequence selected from the group consisting of:
(a) the amino acid sequence set forth in SEQ ID NO: 2, 4, 6, 8, 10, or 12;
(b) the amino acid sequence encoded by the nucleotide sequence set forth in SEQ ID NO: 1, 3, 5, 7, 9, or 11;
(c) an amino acid sequence having at least 95% amino acid sequence identity to at least one amino acid sequence selected from the group consisting of SEQ ID NOS: 2, 4, 6, 8, 10, and 12, wherein said polypeptide comprises AHAS activity;
(d) an amino acid sequence having at least 95% amino acid sequence identity to at least one amino acid sequence selected from the group consisting of SEQ ID NOS: 2, 4, 6, 8, 10, and 12, wherein said polypeptide comprises an asparagine at amino acid position 579 or equivalent position and comprises herbicide-tolerant AHAS activity; and
(e) an amino acid sequence having at least 95% amino acid sequence identity to at least one amino acid sequence selected from the group consisting of SEQ ID NOS: 2, 4, 6, 8, 10, and 12, wherein said polypeptide comprises herbicide-tolerant AHAS activity.
